(19) United States
(12) Patent Application Publication
(10) Pub. No.: US 2022/0349008 A1
(43) Pub. Date: Nov. 3, 2022

(54) NOVEL GENETIC MARKERS FOR POSTURAL ORTHOSTATIC TACHYCARDIA SYNDROME (POTS) AND METHODS OF USE THEREOF FOR DIAGNOSIS AND TREATMENT OF THE SAME

(71) Applicant: THE CHILDREN'S HOSPITAL OF PHILADELPHIA, Philadelphia, PA (US)

(72) Inventors: Hakon Hakonarson, Malvern, PA (US); Huiqi Qu, Drexel Hill, PA (US)

(73) Assignee: THE CHILDREN'S HOSPITAL OF PHILADELPHIA, Philadelphia, PA (US)

(21) Appl. No.: 17/641,650

(22) PCT Filed: Sep. 9, 2020
(86) PCT No.: PCT/US2020/050017

§ 371 Date: Mar. 9, 2022

Related U.S. Application Data
(60) Provisional application No. 62/897,740, filed on Sep. 9, 2019.

(51) Int. Cl. C12Q 1/6883

(52) U.S. Cl. CPC C12Q 1/6883; C12Q 1/6869

(57) ABSTRACT

Compositions and methods for the diagnosis and treatment of POTS are disclosed. In one aspect, a method for identifying a human subject having a predisposition for, or having, postural orthostatic tachycardia syndrome (POTS) is provided. An exemplary embodiment comprises obtaining a nucleic acid sample from said subject; detecting a nucleic acid having one or more single nucleotide polymorphisms (SNPs) in the locus encoding PPP1R12B selected from rs12741415 and/or GSA-rs116062217, or a SNP in linkage disequilibrium with one or more of the SNPs, by contacting the nucleic acid sample with a probe or primer of sufficient length and composition to detect the SNP; and identifying the subject as having a predisposition for POTS if one or more SNPs are identified.

This application claims priority to U.S. Provisional Patent Application No. 62/897,740 filed Sep. 9, 2019, the entire disclosure being incorporated herein by reference.

### FIELD OF THE INVENTION

The present invention relates to the fields of POTS and genetic testing. More specifically, the invention provides compositions and methods for the diagnosis and treatment of POTS and other disorders of the autonomic nervous system.

### BACKGROUND

Several publications and patent documents are cited throughout the specification in order to describe the state of the art to which this invention pertains. Each of these citations is incorporated by reference herein as though set forth in full.

Postural orthostatic tachycardia syndrome (POTS) is a disorder of the autonomic nervous system which controls involuntary body functions. In POTS, the nerves that regulate blood flow do not signal correctly causing orthostatic intolerance. As a result, blood stays in the lower part of the body upon positional change, such as standing. A healthy person's heart rate is usually 70 or 80 beats per minute when lying down. Normally, the heart rate rises by about 10 to 15 beats per minute upon standing. In POTS patients, the heart rate typically increases by 30 to 50 beats per minute or more, which may lead to dizziness and fainting. Other symptoms can include blurry vision, nausea, diarrhea, and constipation palpitations, shortness of breath, sweating, weakness or “heaviness” in the lower legs, brain fog, fatigue, chest pain, anxiety, headaches, insomnia, and co-morbid conditions such as features of Ehlers-Danlos disease. The syndrome affects about 0.2% to 1.0% US population, is more common in females and often starts in teenage years, e.g., 12-14 years of age.

Several diseases and conditions have been associated with POTS. These include anemia, autoimmune diseases, e.g., Sjogren's syndrome or lupus, chronic fatigue syndrome, diabetes and prediabetes, Ehlers-Danlos (a muscle and joint condition), infections such as mononucleosis, Lyme disease, or hepatitis C, multiple sclerosis, and mitral valve prolapse. Investigators have described the presence of several neural receptor antibodies and non-specific autoimmune markers in POTS, yet no definitive treatment regimens are currently available.

Diagnosing POTS is difficult and typically entails use of a tilt-table test. The patient is asked to lie down on a table and strapped in to prevent falling. The table is initially in the horizontal position and is then slowly moved to vertical to simulate standing up as the patient's heart rate is monitored. There is currently no specific therapy or cure for POTS. Clearly, a need exists for improved diagnostic approaches and treatment protocols for ameliorating the symptoms this syndrome.

### SUMMARY

In accordance with the present invention compositions and methods for diagnosis and treatment of POTS are provided that alleviate the paroxysmal changes in orthostatic blood pressure symptoms in this syndrome which employ agents directly tailored to the mechanism of this disease.

In one aspect, a method for identifying a human subject having a predisposition for, or having, postural orthostatic tachycardia syndrome (POTS) is provided. An exemplary embodiment comprises obtaining a nucleic acid sample from said subject; detecting a nucleic acid having one or more single nucleotide polymorphisms (SNPs) in the locus encoding PPPIR12B selected from rs12741415 and/or GSA-rs116062217, or a SNP in linkage disequilibrium with one or more of the SNPs, by contacting the nucleic acid sample with a probe or primer of sufficient length and composition to detect the SNP; and identifying the subject as having a predisposition for POTS if one or more SNPs are identified. Another embodiment comprises obtaining a nucleic acid sample from said subject; detecting a nucleic acid having at least one single nucleotide polymorphisms (SNPs), rs6917603, which maps to the intronic region of the gene ZNRD1ASP (zinc ribbon domain containing 1 antisense, pseudogene), or a SNP in linkage disequilibrium with one or more of the SNPs, by contacting the nucleic acid sample with a probe or primer of sufficient length and composition to detect the SNP; and identifying the subject as having a predisposition for POTS if one or more SNPs are identified. The method can also include detection of additional SNPs selected from those listed in FIG. 3. In other aspects, the method comprises administering at least one an agent useful to treat POTS, said agent being effective to reverse adverse functional consequences of variants impacting the PPP1R12B gene, wherein said agent is an antibody or functional fragment thereof, an siRNA, an antisense oligonucleotide, or a small molecule.

In another embodiment a method for diagnosing and treating POTS in a human subject is disclosed. An exemplary method comprises detecting whether at least one single nucleotide polymorphism (SNP), rs12741415 and/or rs6917603 is present in a nucleic acid sample from the subject, diagnosing the subject with POTS when the presence of at least one SNP is detected; and administering an effective amount of an agent useful for the treatment of POTS. The method can also include detection of one or more SNPs listed in FIG. 3. In certain aspects of the method, the step of detecting the presence of the SNP is performed using a process selected from detection of specific hybridization, measurement of allele size, restriction fragment length polymorphism analysis, allele-specific hybridization analysis, single base primer extension reaction, and sequencing of an amplified polynucleotide. The nucleic acid could be obtained from any source, including but not limited to blood, urine, serum, gastric lavage, cerebral spinal fluid, brain cells, mononuclear cells, cardiac cells, muscle cells, fetal cells in maternal circulation, or body tissue. Also provided is a kit for practicing any of the foregoing methods.

Symptoms to be alleviated include, without limitation, one or more of orthostatic hypotension, large positional changes in heart rate, sensation of fainting or any of the complications and comorbid features of POTS, such as psychological symptoms of anxiety, mood swings, depression, symptoms of Ehlers-Danlos disease, mast cell activation syndrome, vasovagal syncope, dysautonomia, irritable bowel syndrome, insomnia, chronic headaches, chronic fatigue syndrome or fibromyalgia, respectively, comprising administering one or more agents useful in treating POTS. Agents useful for this purpose include, for example, an antibody or functional fragment thereof, an siRNA, an antisense oligonucleotide, or a small molecule, salt tablets, fludrocortisone, pyridostigmine, midodrine, a beta blocker and use of high-high medical compression stockings

### BRIEF DESCRIPTION OF THE DRAWINGS

FIG. 1: Table showing genetic relationship of POTS subjects obtained during recruitment and biobanking of POTS samples. The patients were studied as two independent cohorts, a family cohort and a case control cohort. The Family cohort includes 114 POTS cases (including 28 males and 86 females) from 100 complete families. 62 unaffected siblings from these families were also included in this study. Among these patients, comorbidities were seen in 18 unrelated patients. The case control cohort includes 207 unrelated cases (including 53 males and 154 females) vs. 4328 ethnicity matched controls. Among the 207 cases, comorbidities were seen in 26 unrelated patients.

FIG. 2: A chart showing the median age of diagnosis of the patients in the cohort. The POTS patients were diagnosed at age from 1.75 years old to 24.5 years old, and the median age was 15.6 years old.

FIG. 3: The transmission disequilibrium test (TDT) results. The family cohort was used as the discovery cohort, which was tested by transmission disequilibrium test (TDT) immune to population stratification. By this approach, we avoided potential selection bias in the case control cohort although we selected controls by matching ethnicity. Kinship between family members in the family cohort was validated by identity by descent (IBD) analysis based on the auto-chromosomal genotyping data.

FIG. 4: The PPP1R12B locus identified in the GWA study. As shown by the TDT test results of 612,302 autosomal SNPs, one SNP rs12741415 showed genome-wide significance (P=3.39×10⁻⁹). Another SNP GSA-rs116062217˜43 kb away and in LD with rs12741415 (r²=0.839) had P=9.33E-08 in the TDT cohort, and P=8.49E-13 in the case control cohort. Both SNPs, rs12741415 and rs116062217, map to the intronic region of the protein phosphatase 1 regulatory subunit 12B gene (PPP1R12B) (mapping of the PPP1R12B locus was made by the LocusZoom web-based platform²).

FIG. 5: Expression profile of the PPP1R12B gene. PPP1R12B encodes the large regulatory subunit of myosin phosphatase, which is myosin phosphatase target (MYPT) and the small regulatory subunit M20³. The gene has the highest expression in heart tissue⁴. It plays critical roles in the dephosphorylation of the regulatory light chain of myosin II and muscle relaxation⁵.

FIG. 6: The role of PPP1R12B in vascular smooth muscle contraction. PPP1R12B encodes the large regulatory subunit MYPT and the small regulatory subunit M20 of myosin light chain phosphatase (MLCP)³. Vascular smooth muscle contraction is regulated through reversible phosphorylation-dephosphorylation of the regulatory light chain (LC20) of myosin II, mediated by Myosin light chain kinase (MLCK) and MLCP respectively. The balance of MLCK and MLCP activities determines the steady-state of vascular smooth muscle. [The figure was adopted from the review article by Cole WC, Welsh DG. Arch Biochem Biophys. 2011 June 15;510(2):I60-73.]

FIGS. 7A-7B: FIG. 7A: The association of the PPP1R12B SNPs with POTS. As shown by the TDT test results of 612,302 autosomal SNPs, one SNP rs12741415 showed genome-wide significance (P=3.39×10⁻⁹). Consequently, the association was replicated in the case-control cohort with P=1.10×10⁻¹². The combined P value of the two cohorts was P=1.79×10⁻¹⁹. It is worth to note that, according to the NCBI dbSNP record and the HapMap data, this locus has 3 alleles, i.e. the ancestral allele is G, and the variant allele is A or T. No A/A homozygote was seen in the HapMap European population. In our case-control cohort, the frequencies of genotyping calls were: in controls 0/AG/GG=71/2132/2125; in cases 0/AG/GG=3/50/154. In this case, we can take that the A allele has been correctly called, though some GG could be GT and “0” could be TT. The association test in the case control cohort by assuming A allele as the dominant effect had P=4.26×10⁻¹³, OR (95% CI)=0.3280 (0.2373, 0.4535). In addition, we examined the PPP1R12B gene region with 100 kb flanking region on each side. Another SNP GSA-rs116062217 —43 kb away and in LD with rs12741415 (r²=0.839) had P=9.33E-08 in the TDT cohort, and P=8.49E-13 in the case control cohort. FIG. 7B: Table legend: Table shows results combined analysis the two batches of TDT samples—data from FIG. 7A.

FIG. 8: In this study additional loci were identified wherein loci with Mendelian errors>3 or missing rate>5% were removed from further analysis (the gray rows in the table). One SNP rs6917603 at the HLA class I region didn't show genome-wide significance in the TDT cohort, but reached genome-wide significance when combining the TDT cohort and the case control cohort (P=1.82E-09).

FIG. 9: Mapping of the HLA class I region around the SNP rs6917603 in the TDT cohort. rs6917603 maps to the intronic region of the gene ZNRD1ASP (zinc ribbon domain containing 1 antisense, pseudogene). The locus mapping was made by the LocusZoom web-based platform².

FIG. 10: Mapping of the HLA class I region around the SNP rs6917603 in the case control cohort. rs6917603 maps to the intronic region of the gene ZNRD1ASP (zinc ribbon domain containing 1 antisense, pseudogene). The locus mapping was made by the LocusZoom web-based platform.

FIG. 11A-11B: The realtime PCR results of PPP1R12B in LCLs. FIG. 11A shows the detection of PPP1R12B. The samples 1-3 are males; 4-6 are females. FIG. 11B shows the negative (no template) control.

Table 1: 380 P/LP (pathogenic/likely pathogenic) mutations identified in POTS subjects.

### DETAILED DESCRIPTION

Postural orthostatic tachycardia syndrome (POTS) causes significant functional impairment and psychological distress to the patients, and affects about 0.2% to 1.0% US population. The pathophysiology of POTS has been described as being a failure of ability to maintain vascular tone with a compensatory tachycardia. There is associated chronic orthostatic intolerance and severe debilitating symptoms. However, the actual genetic mechanism of POTS is not understood.

Although the genetic cause of POTS is poorly understood, numerous reports of families with a history of POTS, and a high rate of co-morbid autoimmune disease, suggests that inherited factors play a role in the development of POTS. Additionally, patients may develop POTS after a trauma or exposure to a viral illness. In some cases, patients may develop POTS while recovering from a Sars-Cov2 infection. This suggests that some environmental factors may be linked to the triggering of POTS. Identification of patient with an increased risk of triggering POTS prior to their development of POTS has the potential to allow these patient to avoid a trigger.

A genome wide association study (GWAS) was performed to determine the genetic underpinnings of POTS. We recruited 465 patients diagnosed with POTS and available family members. We obtained a biological sample (blood or saliva) for DNA isolation and performed GWAS analysis using the Illumina Infinium Global Screening Array. Transmission disequilibrium testing (TDT) in available family trios was also conducted. With Bonferroni correction, significance was defined as 7.14×10⁻⁸. TDT analysis was performed in 102 family triads, and case control analysis in 300 patients versus 4,328 ethnicity-matched controls. A non-coding variant in the protein phsphorylase 1 regulatory subunit 12B(PPP1R12B) for myosin, located on chromosome 1q32.1, demonstrated significance at 5.44E-16. This was replicated in both independent TDT and case-control analysis. Also, a non-coding variant of human zinc ribbon domain antisense RNA 1 (ZNRD1-AS1), in the major histocompatibility class (MHC) I region located on chromosome 6, demonstrated significance at 1.82×10⁻⁹. PPP1R12B, which dephosphorylates the regulatory light chain of myosin II, also provides a clinically relevant gene target for POTS, as myosin is expressed in multiple locations in the body, including myocardium and vascular smooth muscle.

ZNRD1-AS1 is a long non-protein coding RNA and resides in the HLA class 1 region. Many patients have onset of symptoms after an infection or concussion, suggesting an autoimmune etiology involving the MHC region. While these specific loci are considered to be non-coding in humans, these variants could interfere with RNA expression of other genes.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu.

As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“POTS-associated SNP or specific marker” is a SNP or marker which is associated with an increased or decreased risk of developing POTS and found in lesser frequency in normal subjects who do not have this disease. Such markers may include but are not limited to nucleic acids, proteins encoded thereby, or other small molecules.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

“Linkage” describes the tendency of genes, alleles, loci or genetic markers to be inherited together as a result of their location on the same chromosome, and is measured by percent recombination (also called recombination fraction, or ⊙) between the two genes, alleles, loci or genetic markers. The closer two loci physically are on the chromosome, the lower the recombination fraction will be. Normally, when a polymorphic site from within a disease-causing gene is tested for linkage with the disease, the recombination fraction will be zero, indicating that the disease and the disease-causing gene are always co-inherited. In rare cases, when a gene spans a very large segment of the genome, it may be possible to observe recombination between polymorphic sites on one end of the gene and causative mutations on the other. However, if the causative mutation is the polymorphism being tested for linkage with the disease, no recombination will be observed.

“Centimorgan” is a unit of genetic distance signifying linkage between two genetic markers, alleles, genes or loci, corresponding to a probability of recombination between the two markers or loci of 1% for any meiotic event.

“Linkage disequilibrium” or “allelic association” means the preferential association of a particular allele, locus, gene or genetic marker with a specific allele, locus, gene or genetic marker at a nearby chromosomal location more frequently than expected by chance for any particular allele frequency in the population. Once a known SNP is identified, SNPs in linkage disequilibrium (also termed LD) may be identified via commercially available programs. For example, on the world wide web at analysistools.nci.nih.gov/LDlink/?tab=ldproxy. First, the LDproxy tab is selected. The reference rs number is entered, the r2 tab and the population of interest are selected and the SNPs in LD identified upon clicking on the “calculate” tab. A plot of surrounding area is revealed and a table with the SNPs in LD (with r2 values) is shown.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with POTS. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any POTS specific marker nucleic acid, but does not hybridize to other nucleotides. Also polynucleotide which “specifically hybridizes” may hybridize only to a POTS-specific marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tm=81.5° C.+16.6 Log [Na+]+0.41(% G+C)−0.63(% formamide)−600/#bp in duplex.

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tm of the hybrid. Wash conditions should be as stringent as possible for the degree of identity of the probe for the target. In general, wash conditions are selected to be approximately 12-20° C. below the Tm of the hybrid. The nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20, at least about 30, at least about 40 or about 50 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and use of the method. For example, for diagnostic applications, depending on the complexity of the target sequence, the oligonucleotide probe typically contains 10, 15-25, 30, 50 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 10, 15-25, 30, 50 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template primer complex for the synthesis of the extension product. Polymerase chain reaction (PCR) has been described in U.S. Pat. Nos. 4,683,195, 4,800,195, and 4,965,188, the entire disclosures of which are incorporated by reference herein.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting DENN/D1B mRNA, for example, may be between 15-35 nucleotides in length, and more typically about 21 nucleotides in length. Exemplary siRNA sequences effective for down-modulating expression of the POTS associated genes can be readily obtained from the above identified commercial sources.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the POTS specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the POTS specific marker encoding nucleic acid. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein.

This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15, greater than 20 nucleotides long or greater than 30 nucleotides long.

“Sample” or “patient sample” or “biological sample” generally refers to a sample which may be tested for a particular molecule, preferably an POTS specific marker molecule, such as a marker shown in the tables provided below. Samples may include but are not limited to cells, body fluids, including blood, serum, plasma, CNS fluid, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays described hereinbelow.

Methods of Using POTS-Associated SNPS for Diagnosing a Propensity for the Development of, or the presence of POTS

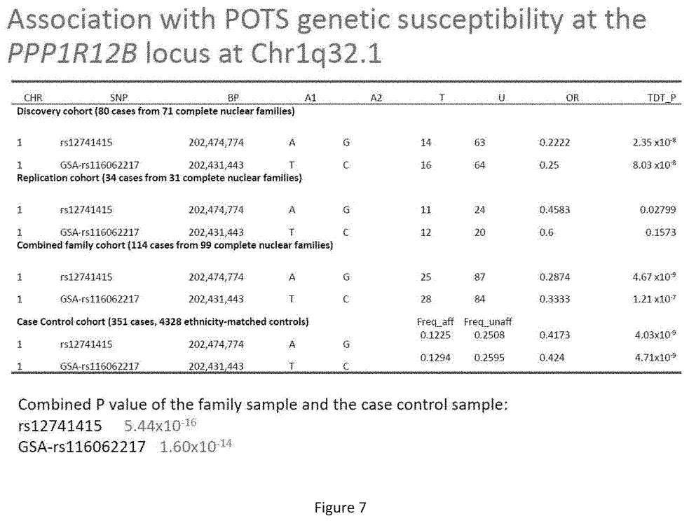

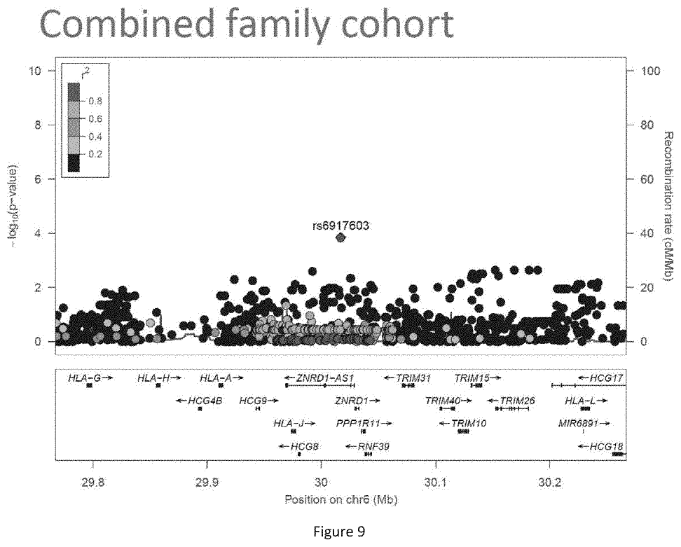

Nucleotides comprising POTS-associated single nucleotide polymorphisms (SNPs) as described in FIG. 7, and/or FIG. 9 may be used for a variety of purposes in accordance with the present invention. For example, POTS-associated SNP-containing DNA, RNA, or fragments thereof may be used as probes or primers to detect the presence of and/or expression of POTS-associated SNPs, or SNPs in linkage disequilibrium with one or more of the POTS-associated SNPs. Methods in which SNP-containing nucleic acids may be utilized as probes or primers include, but are not limited to: (1) in situ hybridization; (2) Southern hybridization (3) northern hybridization; and (4) assorted amplification reactions such as polymerase chain reactions (PCR) or quantitative PCR (qPCR).

Further, assays for detecting POTS-associated SNPs or the proteins encoded thereby may be conducted on any type of biological sample, including but not limited to body fluids (including blood, CNS, bronchial lavage, sputum, serum, gastric lavage, urine), any type of cell (such as brain cells, white blood cells, lung cells, fibroblast cells, mononuclear cells) or body tissue.

From the foregoing discussion, it can be seen that POTS-associated SNP containing nucleic acids, vectors expressing the same, POTS-associated SNP containing marker proteins and anti-POTS specific marker antibodies may be used to detect POTS associated SNPs in body tissue, cells, or fluid, and to diagnose, detect, or identify a human subject as having a predisposition for, or having, POTS.

In some embodiments for screening for POTS-associated SNPs, the POTS-associated SNP containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus, any of the aforementioned techniques may be used to detect or quantify POTS-associated SNP marker expression and accordingly, diagnose POTS.

Kits and Articles of Manufacture

Any of the aforementioned SNP-containing nucleic acids can be incorporated into a kit. In some embodiments, the kit comprises one or more nucleic acid molecules comprising a POTS-associated SNP. In some embodiments, the nucleic acid molecule is immobilized on a solid support, such as on a Gene Chip. In some embodiments, the solid support is affixed to the support so that it does not diffuse from the support when placed in solution. In some embodiments, the kit further comprises an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

Methods of Using POTS-Associated SNPS for Development of Therapeutic Agents

Since the SNPs identified herein have been associated with the etiology of POTS, methods for identifying agents that modulate the activity of the genes and their encoded products containing such SNPs should result in the generation of efficacious therapeutic agents for the treatment of this condition.

The PPP1R12B (also known as Protein Phosphatase 1 Regulatory Subunit 12B; Protein Phosphatase 1, Regulatory (Inhibitor) Subunit 12B; Myosin Phosphatase-Targeting Subunit 2; Myosin Phosphatase Target Subunit 2; MYPT2; Protein Phosphatase 1, Regulatory Subunit 12B; Myosin Phosphatase Regulatory Subunit; Myosin Phosphatase, Target Subunit 2 and PP1bp55) locus provide suitable targets for the rational design of therapeutic agents which modulate the activity of this protein. Genes residing in the HLA class I region also appear to be affected in POTS. Small peptide molecules corresponding to these regions may be used to advantage in the design of therapeutic agents which effectively modulate the activity of the encoded proteins.

Myosin phosphatase is a protein complex comprised of three subunits: a catalytic subunit (PP1c-delta, protein phosphatase 1, catalytic subunit delta), a large regulatory subunit (MYPT, myosin phosphatase target) and small regulatory subunit (sm-M20). Two isoforms of MYPT have been isolated--MYPT1 and MYPT2, the first of which is widely expressed, and the second of which may be specific to heart, skeletal muscle, and brain. Each of the MYPT isoforms functions to bind PP1c-delta and increase phosphatase activity. This locus encodes both MYTP2 and M20. Alternatively, spliced transcript variants encoding different isoforms have been identified. Related pseudogenes have been defined on the Y chromosome.

Molecular modeling should facilitate the identification of specific organic molecules with capacity to bind to the active site of the proteins encoded by the SNP containing nucleic acids based on conformation or key amino acid residues required for function. A combinatorial chemistry approach will be used to identify molecules with greatest activity and then iterations of these molecules will be developed for further cycles of screening. In certain embodiments, candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China).

Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can be used in the screens.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as brain, neuronal, CNS, blood, heart, GI cells) which have a nonfunctional or altered POTS associated gene. These host cell lines or cells are defective at the polypeptide level. The host cell lines or cells are grown in the presence of drug compound and assessed for a POTS associated cellular parameter. Host cells contemplated for use in the present invention include but are not limited to eucaryotice cells, bacterial cells, fungal cells, insect cells, mammalian cells, and plant cells. The POTS-associated SNP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the ga14 inducible promoter and the PGK constitutive promoter. In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the POTS-associated SNPs of the present invention or functional fragments thereof provide a system in which to screen potential compounds or agents for the ability to modulate the development of POTS. Thus, in one embodiment, the nucleic acid molecules of the invention may be used to create recombinant cell lines for use in assays to identify agents which modulate aspects of aberrant cytokine signaling associated with POTS and aberrant bronchoconstriction. Also provided herein are methods to screen for compounds capable of modulating the function of proteins encoded by SNP containing nucleic acids.

Another approach entails the use of phage display libraries engineered to express fragment of the polypeptides encoded by the SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of SNP containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of POTS-associated SNP containing nucleic acids enables the production of strains of laboratory mice carrying nucleic acids harboring the POTS-associated SNPs of the invention. Transgenic mice expressing the POTS-associated SNP of the invention provide a model system in which to examine the role of the protein encoded by the SNP containing nucleic acid in the development and progression towards POTS. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various processes associated with the POTS phenotype, including autonomic nervous system aberrations and maintenance of proper blood flow. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of POTS-associated SNP containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated POTS-associated SNP genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10′ and 10⁻³. Nonhomologous plasmid-chromosome interactions are more frequent occurring at levels 10⁵-fold to 10² fold greater than comparable homologous insertion. To overcome this low proportion of targeted recombination in murine ES cells, various strategies have been developed to detect or select rare homologous recombinants. One approach for detecting homologous alteration events uses the polymerase chain reaction (PCR) to screen pools of transformed cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformed cells can be increased. Utilizing POTS-associated SNP containing nucleic acid as a targeted insertional cassette provides means to detect a successful insertion as visualized, for example, by acquisition of immunoreactivity to an antibody immunologically specific for the polypeptide encoded by POTS-associated SNP nucleic acid and, therefore, facilitates screening/selection of ES cells with the desired genotype.

As used herein, a knock-in animal is one in which the endogenous murine gene, for example, has been replaced with human POTS-associated SNP containing gene of the invention. Such knock-in animals provide an ideal model system for studying the development of POTS.

As used herein, the expression of a POTS-associated SNP containing nucleic acid, fragment thereof, or an POTS-associated SNP fusion protein can be targeted in a “tissue specific manner” or “cell type specific manner” using a vector in which nucleic acid sequences encoding all or a portion of POTS-associated SNP are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the POTS-associated SNP of the invention may be operably linked to a variety of different promoter sequences for expression in transgenic animals. Such promoters include, but are not limited to airway cell specific promoters, a CMV promoter, a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; and a PGK promoter; for the expression of transgenes in airway smooth muscle cells.

Methods of use for the transgenic mice of the invention are also provided herein. Transgenic mice into which a nucleic acid containing the POTS-associated SNP or its encoded protein have been introduced are useful, for example, to develop screening methods to screen therapeutic agents to identify those capable of modulating the development of POTS.

Pharmaceuticals and Methods of Treatment and Uses

In some embodiments, methods for treating POTS are provided comprising administering an agent useful in the treatment of POTS to a subject having one or more SNPs recited in FIG. 7, or a SNP in linkage disequilibrium with one or more of these SNPs.

In some embodiments, methods for treating POTS in a European subject comprising administering an agent useful in the treatment of POTS to a subject having one or more SNPs described herein or a SNP in linkage disequilibrium with one or more of these SNPs.

In each of the method of treating embodiments described above, the method may further comprise detecting or diagnosing the subject prior to treatment, wherein the detection or diagnosing comprises detecting one or more SNPs recited in FIG. 7 and/or FIG. 9, or a SNP in linkage disequilibrium with one or more of these SNPs.

Treatment of patients suffering from POTS include, without limitation salt tablets, fludrocortisone, pyridostigmine, midodrine, and or a beta blocker. Thigh-high medical compression stockings are also helpful. Certain lifestyle changes, including changes to diet and exercise, have also helped patients manage this condition.

These agents may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, aerosolized, intramuscular, and intraperitoneal routes.

A lipid nanoparticle composition is a composition comprising one or more biologically active molecules independently or in combination with a cationic lipid, a neutral lipid, and/or a polyethyleneglycol-diacylglycerol (i.e., polyethyleneglycol diacylglycerol (PEG-DAG), PEG-cholesterol, or PEG-DMB) conjugate. In one embodiment, the biologically active molecule is encapsulated in the lipid nanoparticle as a result of the process of providing and aqueous solution comprising a biologically active molecule of the invention (i.e., siRNA), providing an organic solution comprising lipid nanoparticle, mixing the two solutions, incubating the solutions, dilution, ultrafiltration, resulting in concentrations suitable to produce nanoparticle compositions.

Nucleic acid molecules can be administered to cells by incorporation into other vehicles, such as biodegradable polymers, hydrogels, cyclodextrins. (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. US 2002130430), biodegradable nanocapsules, and bioadhesive microspheres, or by proteinaceous vectors (O'Hare and Normand, International PCT Publication No. WO 00/53722)

The following example is provided to illustrate certain embodiments of the invention. It is not intended to limit the invention in any way.

### Example I

Discovery of PPP1R12B in POTS as a Critical Mediator of the Effects of Upstream Muscle Expressed Genes Harboring Multiple Pathogenic Mutations

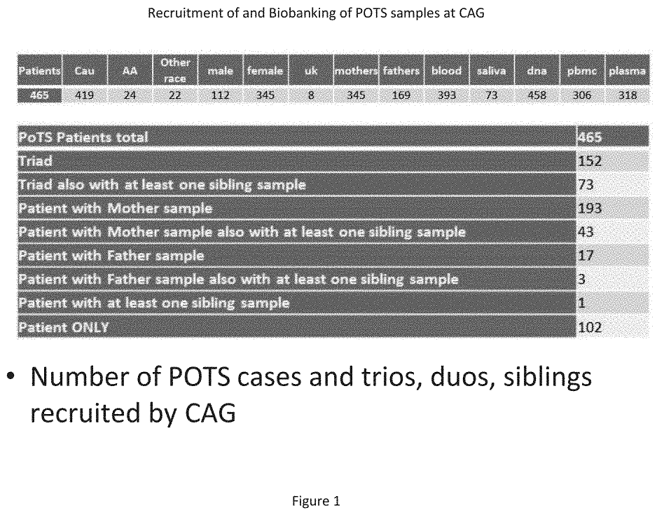

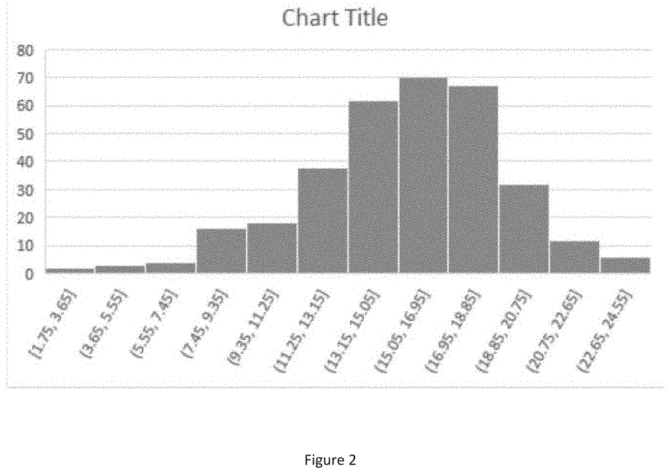

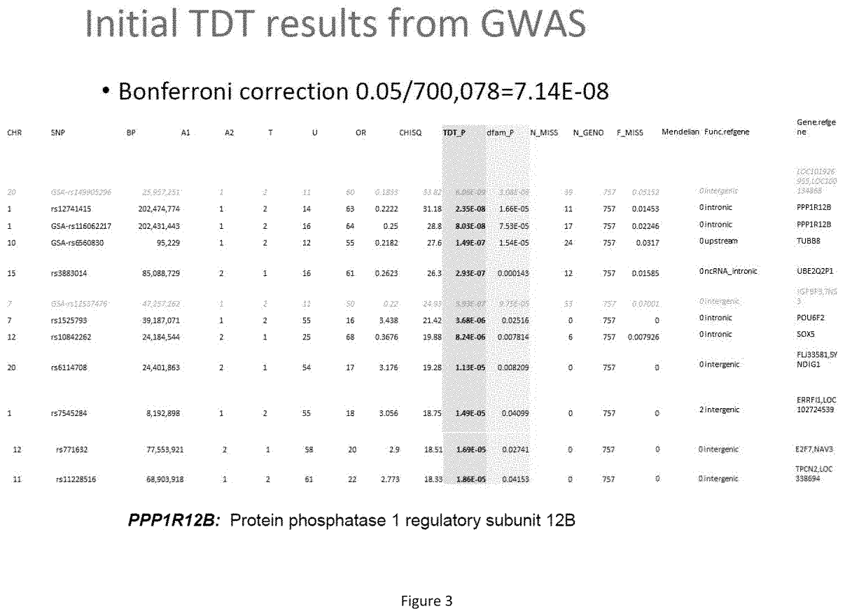

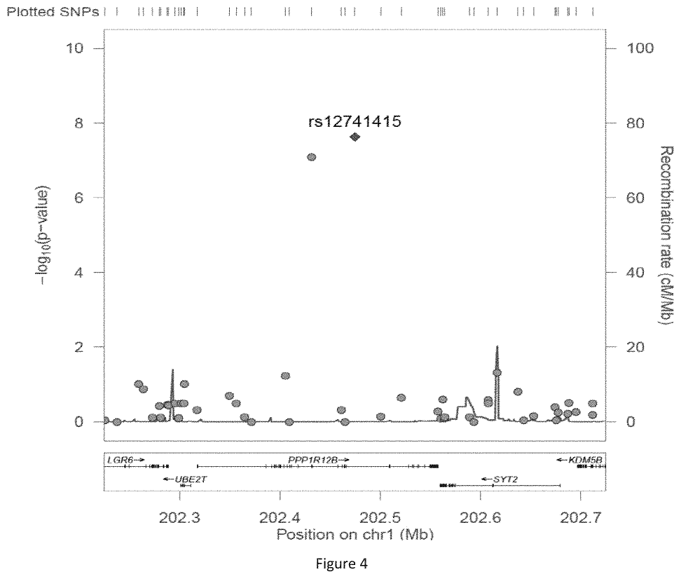

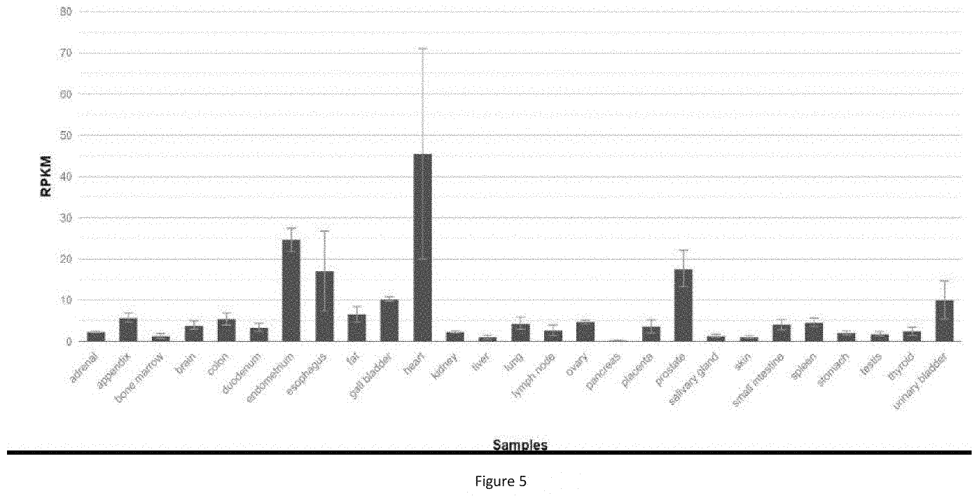

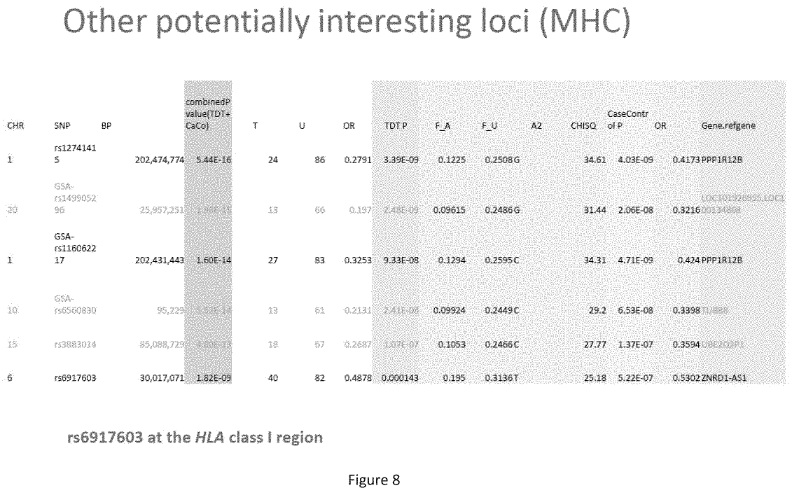

We performed GWAS analysis on 465 POTS patients and we uncovered genome-wide significant association of a tagging SNP (r512741415) on chr1q32.1 in the PPP1R12B gene to POTS disease using 80 cases from 71 complete nuclear families, with replication in 351 cases and 4328 controls, as well as an independent set of 34 cases from 31 complete nuclear families, genotyped on the Illumina GSA array. Combined P value=5.44×10⁻¹⁶ (See FIGS. 1 to 10). PPP1R12B encodes both the large regulatory subunit MYTP2 and the small regulatory subunit M20 of myosin light chain phosphatase (MLCP) and its function is to dephosphorylates the regulatory light chain of myosin II; the gene is expressed in heart muscle and vascular smooth muscle and provides a strong functional/biological candidate for POTS.

We subsequently performed Whole Exome Sequencing (WES) on 303 individuals from 75 of the most informative complete nuclear families, including 87 cases, both parents and 42 unaffected children. We identified 97 pathogenic (P) variants (and additional 201 variants that are likely pathogenic (LP)) based on information from current clinical variant databases (ClinVar, HGMD), that clustered in genes involved with muscle/heart function and neurodevelopmental disease genes. 87 of 88 cases have at least one P or LP variant—this represents—3 fold enrichment for muscular/myopathic genes in POTS compared to the gnomAD database. Six heart conditions are most enriched-cardiomyopathy (both dilated and hypertrophic), LongQT syndrome, Bicuspid Ao valve, TOF and HPLHS. 42 (48%) of the POTS cases have at least one of these heart disease mutations and 8 of the cases have 2 such mutations. A subset of the 465 POTS cases have Ehlers-Danlos Syndrome (EDS) and were also notably enriched. There are 4 rare variants in PPP1R12B that could functionally impact this gene.

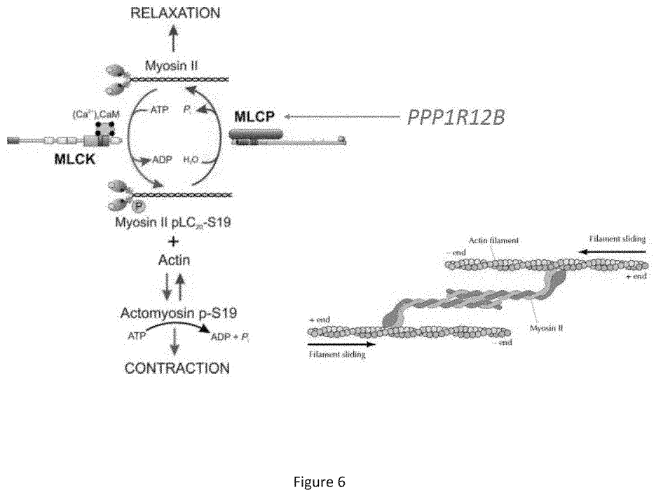

Two nearby genes, UBE2T (causative of Fanconi Anemia and KDM5B (causative of autism, CHD and neurodevelopmental disability) also harbor rare variants. It appears that POTS may be a downstream consequence of multiple mutations associated with multiple genetic conditions where PPPIRI2B is a common pathway for targeted. See FIG. 6. The impact of the upstream P and LP mutations on PPPIRI2B function in cardiac and vascular muscle for drug target validation is also promising.

We observed a robust signal for PPP1R12B, significant— GW significant by TDT and signal is replicated in two independent cohorts, (one TDT and one case-control) We also detected another candidate gene, SLC6A2 (norepinephrine transporter) having a signal of with P value of 10⁻⁵. Applying alternative analysis methods in search for previous candidate genes, revealed the Toll-like receptor pathway may also be significant.

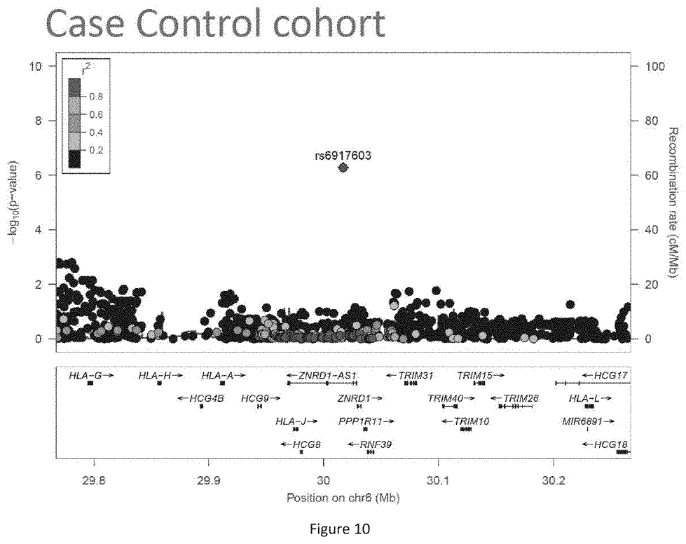

For the first time GWAS show an HLA signal in POTS. See FIGS. 9 and 10. Whole Exome Sequencing (WES) Samples were the most informative TDT families for assessment of rare variants that may explain the signal at the PPP1R12B locus. Signals for TLR locus and the SLC6A2 locus should be replicated shortly.

The identification of multiple POTS cases harboring serious disease-causing mutations provides new therapeutic targets for the treatment of this syndrome. Mutations known to cause different heart conditions where PPP1R12B is a downstream central effector protein involving cardiac muscle relaxation, again providing a new drug target for validation studies.

Some mutations are known to cause/contribute to different neurodevelopmental disorders: These data present an opportunity to pursue further phenotypic characterizations with respect to autism and other NDDs in POTS patients. Notably, the previous GWAS locus at PPP1R12B represents a critical common pathway with protective effects. Our data also reveal a significant association at HLA class I region around the SNP rs6917603 in the TDT cohort. rs6917603 maps to the intronic region of the gene ZNRD1ASP

We have unveiled a potential shared mechanism through which multiple variants and candidate genes could mediate their effects and identify the PPP1R12B gene locus and the ZNRD1ASP gene locus as new drug targets for assessing agents useful to ameliorate POTS symptoms. Understanding the key perturbations in the signaling pathway that are mediated by different gene mutations and reversing them with interventions.

### Example II

Comparison of the Expression of PPP1R12B in Patients with Hypertrophic Cardiomyopathy

We performed data mining of the NCBI's Gene Expression Omnibus (GEO) database. A dataset of expression profiling by array cardiac on tissues from patients with hypertrophic cardiomyopathy was analyzed (GSE36961, https://www.ncbi.nlm.nih.gov/geo/query/acc.cgi?acc=GSE36961). Analysis of this dataset showed that the PPP1R12B levels were correlated with sex, but not with age. The PPP1R12B has higher expression levels in males than females (Tables 1 and 2).

TABLE 1
The expression levels of PPP1R12B are
correlated with sex, but not age

| | Unstandardized | | Standardized | | |
|---|---|---|---|---|---|
| | Coefficients | | Coefficients | t-test | |
| | B | Std. Error | Beta | Value | Sig. |
| (Constant) | 7.368 | 0.113 | | 65.263 | 0 |
| sex | 0.175 | 0.07 | 0.209 | 2.49 | 0.014 |
| age | 0.002 | 0.002 | 0.087 | 1.02 | 0.31 |
| Control | −0.06 | 0.079 | −0.063 | −0.756 | 0.451 |

TABLE 2
The expression levels of PP1R12B in males and females

| sex | Mean | N | Std. Deviation |
|---|---|---|---|
| female | 7.448 | 72 | 0.44 |
| male | 7.61 | 73 | 0.385 |
| Total | 7.529 | 145 | 0.42 |

After identification of the correlation between PPP1R12B levels and sex, we used lymphoblastoid cell lines (LCL) to examine the expression regulation of PPP 1R12B using the PCR primer pairs listed in Table 3.

TABLE 3
PCR primer pairs

| Groups | Primer | Sequence |
|---|---|---|
| 1 | LEFT | GTTCCCTCTGACCTTGCAGA |
| | PRIMER | (SEQ ID NO: 1) |
| | RIGHT | AGCCTGCCTCACATCCTCTA |
| | PRIMER | (SEQ ID NO: 2) |
| 2 | LEFT | CAGGCTGGCTATGAACTCAA |
| | PRIMER | (SEQ ID NO: 3) |
| | RIGHT | GCCACATCAAATGGTGTCTG |
| | PRIMER | (SEQ ID NO: 4) |
| 3 | LEFT | ACACCATTTGATGTGGCTGA |
| | PRIMER | (SEQ ID NO: 5) |
| | RIGHT | ACCTTCCTCCTCCTCTGAGC |
| | PRIMER | (SEQ ID NO: 6) |
| 4 | LEFT | GAAGCCAGGGAGAAGAGGAG |
| | PRIMER | (SEQ ID NO: 7) |
| | RIGHT | CTCCTGAGCTTGCCTCTCTG |
| | PRIMER | (SEQ ID NO: 8) |
| 5 | LEFT | ACCAGTTCCCACCTGCTATG |
| | PRIMER | (SEQ ID NO: 9) |
| | RIGHT | TGACCTCTTCAGAGCCATCA |
| | PRIMER | (SEQ ID NO: 10) |

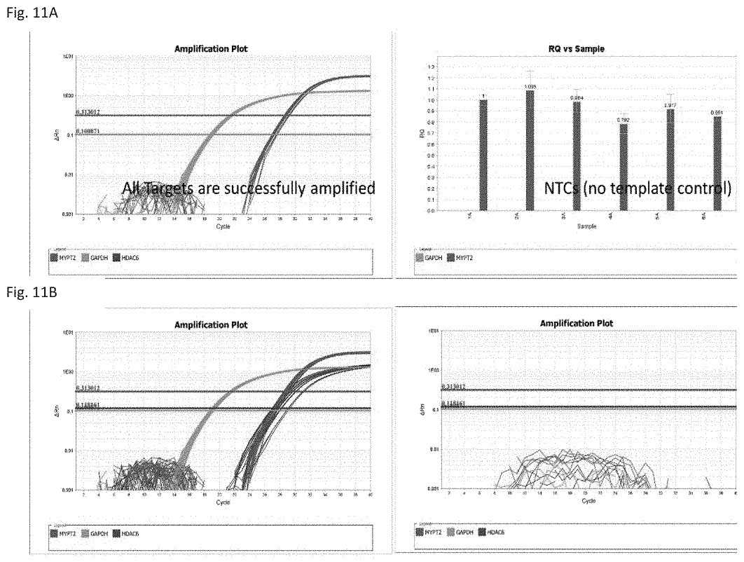

Expression regulation of PPP 1R12B levels were examined using realtime PCR in 6 LCLs, including 3 males and 3 females. The LCL cells were examined to show expression of the PPP1R12B gene and expression levels in males and females were compared. As seen in FIG. 11, expression of PPP 1R12B was higher in male subjects than in female subjects. As male subjects are less likely to develop POTS, the increased expression of PPP 1R12B likely prevents POTS. Therefore, a promising treatment for POTS would involve enhancing the activity of PPP 1R12B, particularly in female subjects.

Considering the protective effects observed in males, a gene residing on the Y chromosome, such as the PPP1R12BP 1 pseudogene, is likely to harbor the explanation of the sex-specificity of POTS. The impact of the PPP1R12BP 1 pseudogene on PPP1R12B function as competition for inhibitory transcription factors is also promising. Moreover, sequence specific molecules could be designed which target these molecules to modulate expression thereof.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for identifying a human subject having a predisposition for, or having postural orthostatic tachycardia syndrome (POTS), comprising,
a) obtaining a nucleic acid sample from said subject;
b) detecting a nucleic acid having at least one single nucleotide polymorphisms (SNPs), in the locus encoding PPP1R12B selected from rs12741415 and/or GSA-rs116062217, and/or at least one SNP in linkage disequilibrium (LD) with the at least one SNPs, or at least one SNPs rs6917603, in the intronic region of the gene ZNRD1ASP, and/or at least one SNP in LD with the SNP, by contacting the nucleic acid sample with a probe or primer of sufficient length and composition to detect the SNP; and
c) identifying the subject as having a predisposition for POTS if one or more SNPs are identified.

2. (canceled)

3. The method of claim 1 or 2, further comprising detection of at least one SNPs at GSA-rs6560830, rs3883014, rs1525793, rs10842262, rs6114708, rs7545284, rs771632, or rs11228516.

4. The method of claim 1, further comprising administering at least one an agent useful to treat POTS.

5. The method of claim 1, wherein said agent reverses adverse functional consequences of variants impacting the PPP 1R12B gene, wherein said agent is an antibody or functional fragment thereof, an siRNA, an antisense oligonucleotide, or a small molecule.

6. The method of claim 1, wherein SNP is in LD with either rs12741415, or GSA-rs116062217, or rs6917603.

7. A method for diagnosing and treating postural orthostatic tachycardia syndrome (POTS) in a human subject, comprising,
a) detecting whether at least one single nucleotide polymorphism (SNP), rs12741415 or rs6917603 is present in a nucleic acid sample from the subject,
b) diagnosing the subject with POTS when the presence of at least one SNP is detected; and
c) administering an effective amount of an agent useful for the treatment of POTS.

8. (canceled)

9. The method of claim 1, wherein the step of detecting the presence of the SNP is performed using a process selected from detection of specific hybridization, measurement of allele size, restriction fragment length polymorphism analysis, allele-specific hybridization analysis, single base primer extension reaction, and sequencing of an amplified polynucleotide.

10. The method of claim 1, wherein the target nucleic acid is DNA.

11. The method of claim 1, wherein the nucleic acid sample is from blood, urine, serum, gastric lavage, cerebral spinal fluid, brain cells, mononuclear cells, cardiac cells, muscle cells, fetal cells in maternal circulation, or body tissue.

12. A method of treating postural orthostatic tachycardia syndrome (POTS) in a patient having any one or more of orthostatic hypotension, large positional changes in heart rate, sensation of fainting or any of the complications and comorbid features of POTS, such as psychological symptoms of anxiety, mood swings, depression, symptoms of Ehlers-Danlos disease, mast cell activation syndrome, vasovagal syncope, dysautonomia, irritable bowel syndrome, insomnia, chronic headaches, chronic fatigue syndrome or fibromyalgia, respectively, comprising administering one or more agents useful in treating POTS.

13. The method of claim 12, wherein the agent is an antibody or functional fragment thereof, an siRNA, an antisense oligonucleotide, or a small molecule.

14. The method of claim 12, wherein said agent is salt tablets, fludrocortisone pyridostigmine, midodrine, a beta blocker and use of high-high medical compression stockings

15. A kit for practicing the method of claim 1.

16. The method of claim 7, wherein the step of detecting the presence of the SNP is performed using a process selected from detection of specific hybridization, measurement of allele size, restriction fragment length polymorphism analysis, allele-specific hybridization analysis, single base primer extension reaction, and sequencing of an amplified polynucleotide.

17. The method of claim 7, wherein the target nucleic acid is DNA.

18. The method of claim 7, wherein the nucleic acid sample is from blood, urine, serum, gastric lavage, cerebral spinal fluid, brain cells, mononuclear cells, cardiac cells, muscle cells, fetal cells in maternal circulation, or body tissue.

19. A kit for practicing the method of claim 7.

20. The method of claim 7, further comprising detection of at least one SNPs at GSA-rs6560830, rs3883014, rs1525793, rs10842262, rs6114708, rs7545284, rs771632, or rs11228516.


SEQUENCE LISTING (10 sequences)

SEQ ID NO: 1 - DNA, 20 bp, Artificial Sequence
  Left PCR Primer
gttccctctgaccttgcaga

SEQ ID NO: 2 - DNA, 20 bp, Artificial Sequence
  Right PCR Primer
agcctgcctcacatcctcta

SEQ ID NO: 3 - DNA, 20 bp, Artificial Sequence
  Left PCR Primer
caggctggctatgaactcaa

SEQ ID NO: 4 - DNA, 20 bp, Artificial Sequence
  Right PCR Primer
gccacatcaaatggtgtctg

SEQ ID NO: 5 - DNA, 20 bp, Artificial Sequence
  Left PCR Primer
acaccatttgatgtggctga

SEQ ID NO: 6 - DNA, 20 bp, Artificial Sequence
  Right PCR Primer
accttcctcctcctctgagc

SEQ ID NO: 7 - DNA, 20 bp, Artificial Sequence
  Left PCR Primer
gaagccagggagaagaggag

SEQ ID NO: 8 - DNA, 20 bp, Artificial Sequence
  Right PCR Primer
ctcctgagcttgcctctctg

SEQ ID NO: 9 - DNA, 20 bp, Artificial Sequence
  Left PCR Primer
accagttcccacctgctatg

SEQ ID NO: 10 - DNA, 20 bp, Artificial Sequence
  Right PCR Primer
tgacctcttcagagccatca